(19) United States
(12) Patent Application Publication
(10) Pub. No.: US 2025/0134752 A1
(43) Pub. Date: May 1, 2025

(54) AN ANTI-MYOPIA VISUAL DISPLAY THERAPY USING SIMULATED MYOPIC BLUR

(71) Applicant: THE UAB RESEARCH FOUNDATION, Birmingham, AL (US)

(72) Inventors: Timothy J. Gawne, Birmingham, AL (US); Thomas T. Norton, Birmingham, AL (US)

(73) Assignee: THE UAB RESEARCH FOUNDATION, Birmingham, AL (US)

(21) Appl. No.: 18/836,134

(22) PCT Filed: Mar. 3, 2023
(86) PCT No.: PCT/US2023/014478

§ 371 Date: Aug. 6, 2024

Related U.S. Application Data
(60) Provisional application No. 63/317,357, filed on Mar. 7, 2022.

(51) Int. Cl. A61H 5/00; G06T 5/70; G06T 5/94

(52) U.S. Cl. CPC A61H 5/00; G06T 5/70; G06T 5/94; A61H 2201/5007; A61H 2201/5043; G06T 2207/10024; G06T 2207/30041

(57) ABSTRACT

The present disclosure relates to techniques for anti-myopia visual display therapy using a simulated myopic blur. In one particular aspect, a method is provided that includes obtaining a pattern for a digital image, selecting a color channel of the digital image, applying a blur effect to the color channel that modifies the digital image to have a simulated blur, and providing anti-myopia visual display therapy to a subject using the modified digital image. The therapy comprises: (i) rendering the modified digital image on a display of a computing device within a visual environment of the subject, or (ii) placing the modified digital image within the visual environment of the subject, based on an optimal viewing time.

### CROSS-REFERENCE TO RELATED APPLICATIONS

This application claims priority to U.S. Provisional Application No. 63/317,357, filed Mar. 7, 2022, which is incorporated by reference herein in its entirety for all purposes.

### STATEMENT OF GOVERNMENT SUPPORT

The invention was made with government support under EY028578 awarded by the National Eye Institute (NEI). The government has certain rights in the invention.

### FIELD

The present disclosure relates to refractive errors of the eye, and in particular to techniques for anti-myopia visual display therapy using a simulated myopic blur.

### BACKGROUND

It has been well established that a feedback mechanism in the postnatal growing eye in humans and other species uses visual (optical) cues to evaluate the focus of the eye and to regulate the rate of axial elongation to achieve, and then actively maintain, sharp focus on the retina through childhood and into at least early adulthood. If the eye is too short for its optics, it is termed hyperopic, and elongation is accelerated; if the eye is too long, it is termed myopic, and elongation is retarded until the increasing focal length of the growing eye moves the focal plane back onto the retina. This process is termed emmetropization. Maintaining emmetropia is critically important for achieving good acuity. In a 24-mm human eye, a mismatch of as little as 175 μm between the location of the focal plane, where images are in focus, and the retina, where images are detected, causes a 0.5-diopter (D) refractive error which reduces visual acuity from 20/20 to about 20/30 (US units; these levels of visual acuity would be represented as from 6/6 to 6/9.5 in most countries using the metric system).

In contrast to perceptual visual acuity, the emmetropization mechanism does not require the central fovea, where acuity is highest, but appears to depend on the peripheral retina. Indeed, emmetropization is primarily a local process that does not require the rest of the brain. A region of retina evaluates visual cues over time, and then a signaling cascade through the retinal pigment epithelium and choroid causes the underlying sclera to become either more or less extensible, thus adjusting the growth of the eye to maintain the foveal retina at the focal plane. The localized retinal control has been demonstrated in chicks, tree shrews, and non-human primates with the use of diffusers or lenses that cover only part of the visual field, where only the part of the sclera adjacent to the retina receiving degraded images elongates. However, the precise nature of the visual cues used by the retina to guide emmetropization has remained unclear.

### SUMMARY

Embodiments are directed to a model of how the self-correcting “emmetropization” mechanism uses wavelength cues to control the refractive state of the human eye. Based on this model, techniques are disclosed herein for a visual display therapy using a simulated myopic blur to prevent or slow myopia development.

In some embodiments, a method is provided that includes: obtaining a pattern for a digital image, selecting a color channel of the digital image, applying a blur effect to the color channel that modifies the digital image to have a simulated blur, and providing anti-myopia visual display therapy to a subject using the modified digital image. The therapy comprises: (i) rendering the modified digital image on a display of a computing device within a visual environment of the subject, or (ii) placing the modified digital image within the visual environment of the subject, based on an optimal viewing time.

In some embodiments, the pattern is a high-contrast pattern of objects on a solid background, which generates multiple black-white edges within the digital image.

In some embodiments, the color channel is selected based on a model of structure and function of an eye that demonstrates how a combination of wavelengths of light and optical defocus regulates growth of the eye.

In some embodiments, the color channel selected is a short wavelength channel.

In some embodiments, the short wavelength channel is the blue channel.

In some embodiments, the blur effect is applied to the color channel in a predetermined amount determined based on a viewing distance and/or display or image size to be viewed by the subject.

In some embodiments, the modified digital image is rendered on the display of the computing device in combination with other images unrelated to the therapy.

In some embodiments, the optimal viewing time is determined based on a present level of refractive error of an eye of the subject.

In some embodiments, the optimal viewing time is periodically through a day.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 shows an emmetropic eye in accordance with various embodiments. Light rays (green solid lines) from a distant object enter the cornea at the front of the eye and are focused at a focal plane by the cornea and lens. The sclera is the outer coating of the eye. The distance from the front of the cornea to the front of the sclera is the axial length, which increases as the eye grows. The retina is the light-sensitive layer of tissue in front of the sclera. The location of the retina is controlled by the axial length

FIGS. 2A and 2B show hyperopic and myopic eyes in accordance with various embodiments. FIG. 2A—In a hyperopic eye, the axial length is shorter than the focal plane; light rays are focused behind the retina and images on the retina are blurred. FIG. 2B—In a myopic eye, the axial length is longer than the focal plane; light rays are focused in front of the retina and images on the retina are blurred.

FIG. 3 shows normalized cone absorbance in an animal model in accordance with various embodiments. The model was developed using data collected from tree shrews and net absorbance is adjusted for the optical filtering properties of the ocular tissues. The short-wavelength sensitive (SWS) cones have an absorbance peak in the blue end of the spectrum, and are insensitive to longer wavelengths. The long-wavelength sensitive (LWS) cones have a broader absorption spectrum, but have a peak sensitivity to yellow light (about 550 nm).

FIGS. 4A and 4B show the eye focuses different wavelengths of light at different distances behind the cornea in accordance with various embodiments. FIG. 4A-blue light (short wavelengths) is focused closer to the cornea. FIG. 4B-red light (long wavelengths) is focused farther from the cornea. These figures exaggerate the wavelength effect for illustration purposes.

FIGS. 5A and 5B show wavelength signals to the emmetropization mechanism in accordance with various embodiments. FIG. 5A-If blue wavelengths are in focus, the red wavelengths are blurred; this signals the emmetropization mechanism that the eye is too short. FIG. 5B-If red wavelengths are in focus (blue blurred), this signals that the growing eye has become too long and growth needs to slow.

FIG. 6 shows two detectors for where light is in focus (SWS and MWS+LWS cones) in accordance with various embodiments.

FIGS. 7A-7C illustrate anti-myopia simulated myopic blur visual display in accordance with various embodiments.

FIGS. 8A and 8B show experimental results from using a tree shrew model in accordance with various embodiments. FIG. 8A-mean refractions as a function of time (average refraction from each animal OD (right eye) and OS (left eye) treated as single data points) for normal open-view colony animals, animals in small cages with closed views, and animals in small cages with simulated myopic blur. FIG. 8B-same, but change in vitreous chamber depth over time.

FIG. 9 shows a flowchart of a process for generating an anti-myopia simulated myopic blur visual display and treating myopia using an anti-myopia simulated myopic blur visual display in accordance with various embodiments.

### DETAILED DESCRIPTION

### I. Overview

As discussed above, a self-correcting feedback mechanism in our childhood adjusts the growth of the eye so that the light-sensitive retina is located where images of the visual world are focused, producing clearly-focused vision (“emmetropia”.) This emmetropization mechanism uses visual cues to determine if the eye is too short (hyperopia) or has grown too long relative to the focal plane (myopia) and adjusts eye growth to move the retina back to emmetropia. However, in many instances, this mechanism fails for a number of number of reasons leaving a person with hyperopia or myopia through adulthood. Myopia is an enormous problem around the world, affecting perhaps more than 1 billion people worldwide. In myopia, the length of the eye is longer than optimal. Even low amounts of myopia raise the risks of developing retinal holes or tears, retinal detachment, choroidal degeneration, glaucoma, cataract, and other potentially blinding conditions caused by the elongated eye. The economic cost of glasses, contact lenses and refractive surgery to correct myopia is many billions of dollars in the US alone but does not remove the risk of blindness because they do not alter the length of the eye so it remains long. Myopia typically develops and increases (progresses) in childhood between the ages of 5 and 15. It has been estimated that the total annual global cost of myopia was $358.7 billion in 2019, and is projected to reach $870 billion in 2050. Slowing myopia development typically requires treatment throughout this extended period and thus must be safe in long-term use. Many companies are trying to develop effective ways to prevent children from developing myopia, or to slow the rate of myopia development so as to reduce the final amount in adulthood. Success using optical (contact lenses, glasses, wavelength filters) or pharmaceutical (eye drops) approaches has been limited. An effective, safe, non-invasive, non-pharmacological and convenient treatment that could be used in the home over many years would be of benefit to billions of people.

To address these limitations and problems, techniques are disclosed herein for controlling the self-correcting feedback mechanism using chromatic simulations of optical blur to alter the chromatic cues. None of the current techniques of myopia control use chromatic simulations of optical blur as the basis of treatment. From the results of studies by the inventors in tree shrews (mammals closely related to primates), a model of how the combination of the wavelengths of light and optical defocus regulates eye growth has been conceived. This model predicts that viewing a pattern with black-white edges in which the short (blue) wavelengths are blurred may stabilize refractive development and prevent or slow myopia development in children. The pattern was tested in tree shrews, which are diurnal mammals closely related to primates, that were placed in a visual environment that produces myopia and found that it prevented the expected myopia. Importantly, this pattern may be displayed using various types of media (e.g., an electronic display of a computing device). No glasses need to be worn and no pharmacological treatment is involved. The pattern simply needs to be viewed periodically during the day. Advantageously, these techniques provide a safe, simple convenient pattern to be viewed daily that could potentially eliminate or greatly reduce the incidence or amount of myopia in millions of people.

One illustrative embodiment of the present disclosure is directed to a method that includes: obtaining a pattern for a digital image, selecting a color channel of the digital image, applying a blur effect to the color channel that modifies the digital image to have a simulated blur, and providing anti-myopia visual display therapy to a subject using the modified digital image. The therapy comprises: (i) rendering the modified digital image on a display of a computing device within a visual environment of the subject, or (ii) placing the modified digital image within the visual environment of the subject, based on an optimal viewing time.

### II. Technical Description

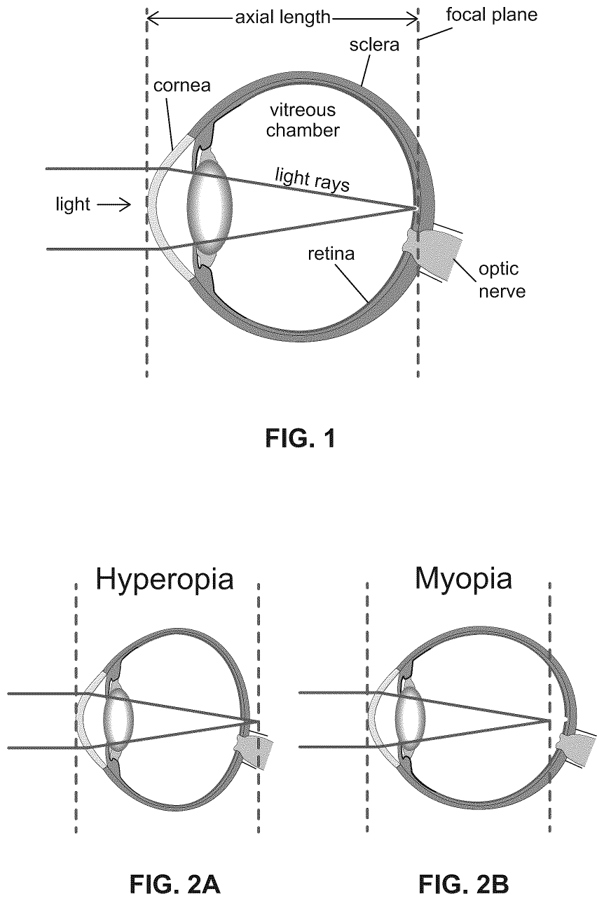

The eye of humans and, indeed, all vertebrates, is a globe with a clear tissue at the front, the cornea, through which light enters the eye. As shown in FIG. 1, the light is focused by the cornea and the lens to a focal plane near the retina at back of the eye. Surrounding the sides and back of the eye is the sclera. The distance from the front of the cornea to the sclera at the back of the eye is the axial length. The retina is the tissue just in front of the sclera that detects light, processes visual images and sends them through the optic nerve to central brain areas that produce visual perception.

For visual images to appear clear (not blurred) the axial length of the eye must position the retina at the focal plane. If the axial length is short relative to the focal plane (FIG. 2A), the images on the retina are blurry; the eye is hyperopic. If the axial length it places the retina behind the focal plane (FIG. 2B), images also are blurry; the eye is myopic.

At birth, most human and animal eyes are hyperopic because the axial length is short relative to the focal plane (FIG. 2A). During postnatal development the eye grows longer and a self-correcting “emmetropization” feedback mechanism uses the out-of-focus images to guide the eye to grow until images are in focus on the retina (emmetropia, (FIG. 1)) and then controls further growth to keep the retina at the focal plane so images remain in focus. It is still not understood what aspects of the visual images are used by the emmetropization mechanism, but recent studies have found that the wavelength of light plays an important role. Outdoor light, and most indoor illumination, contains light of many wavelengths in the range of 400 to 700 nanometers that is visible to humans and other mammals.

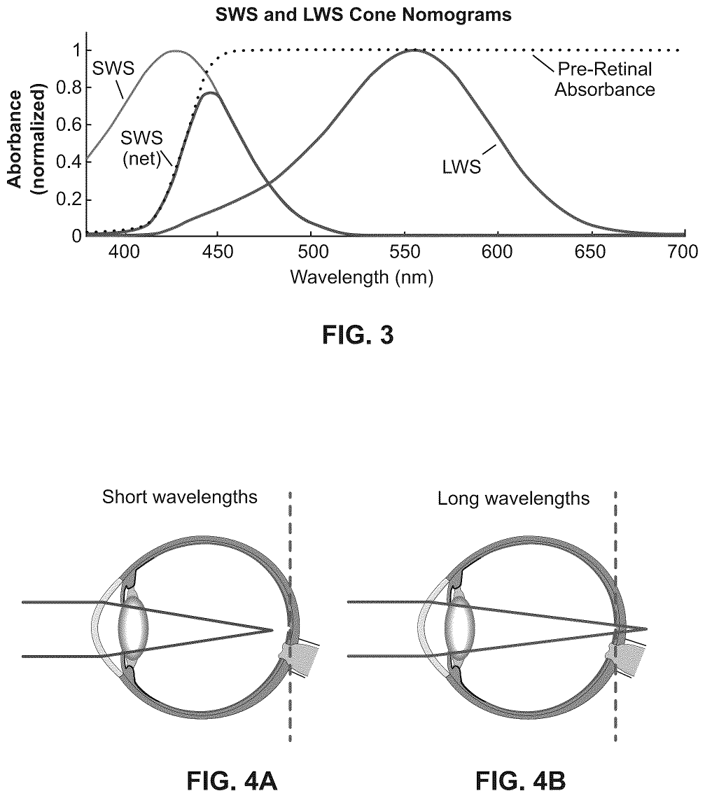

Light is detected because it is absorbed by the photopigments of the cones, the sensory cells in the retina. As shown in FIG. 3, there are two types of cones in most mammals, the SWS cones that preferentially absorb and detect blue light, and LWS that preferentially detect red light. Both types of cones are present throughout the retina.

There also are two additional photopigments in the retina: rhodopsin in the low-light sensitive rods, and melanopsin in the intrinsically photosensitive retinal ganglion cells. However, these two pigments are not thought to be important for high-acuity vision in bright light, so our model ignores them. Also, most humans have a third middle-wavelength sensitive (MWS) cone photopigment. The peak of the MWS absorbance is close to that of the LWS photopigment and the profile of the MWS photopigment overlaps extensively with that of the LWS cones. Dichromatic humans that only have two photopigments, like the tree shrew, emmetropize normally. Thus, for the purposes of emmetropization, the human system is essentially the same as for tree shrews using cone photopigments that absorb at long, versus short wavelengths and predict that the model and the experimental results from tree shrews will generalize to humans.

Outdoor lighting and most indoor lighting contain many wavelengths. The eye focuses different wavelengths (colors) of light (long wavelength/red, and short wavelength/blue) at different distances behind the cornea. Blue wavelengths (FIG. 4A) are in focus nearer the cornea than are red wavelengths (FIG. 4B), a property known as longitudinal chromatic aberration (LCA).

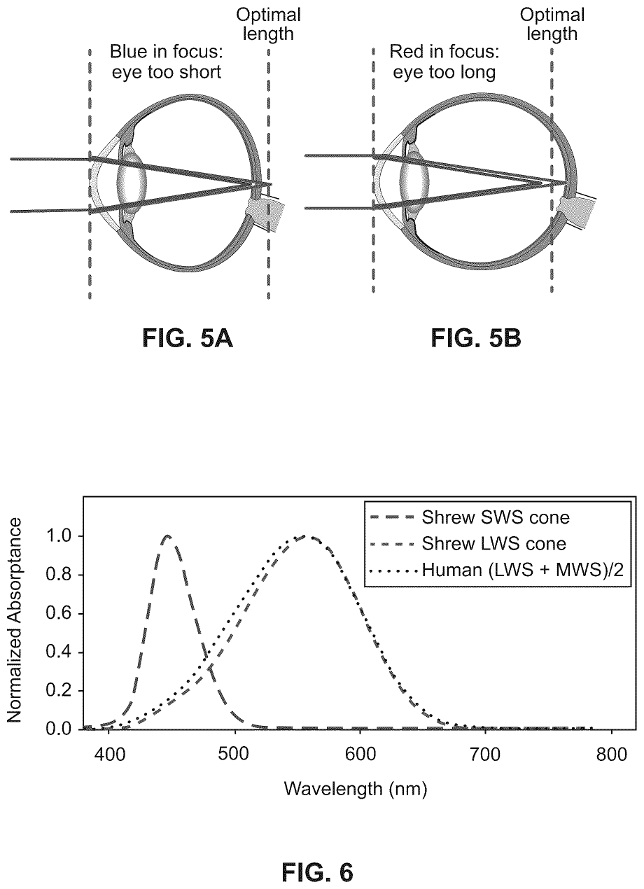

The inventors' studies have found evidence that the emmetropization mechanism uses some aspect of LCA to maintain the axial length within a narrow range. If the blue wavelengths are in focus (FIG. 5A), the red wavelengths are out of focus; this is a cue that the eye is too short and should increase its elongation rate. If the red wavelengths are in focus on the retina (blue out of focus, FIG. 5B), this is a cue that the growing eye has become too long for its own optics and needs to slow its normal postnatal axial elongation rate. Based on these studies, a model (shown in FIG. 6) may be developed in which an opponent dual-detector spectral drive system utilizes longitudinal chromatic aberration to guide normal emmetropization in dichromatic and tri-chromatic species such as humans The challenge was whether LCA could be directly manipulated in accordance with the model to control the emmetropization mechanism.

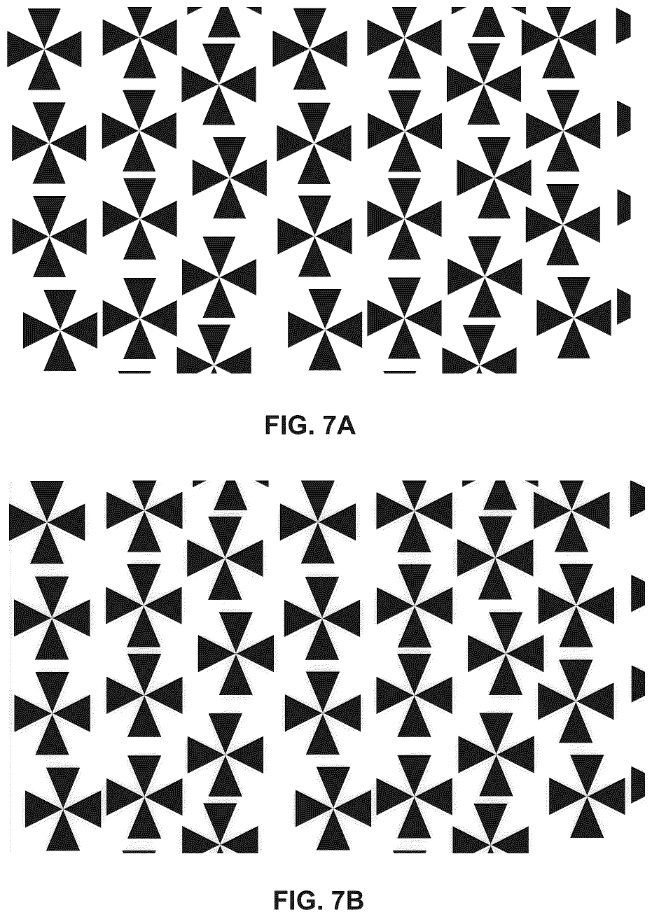

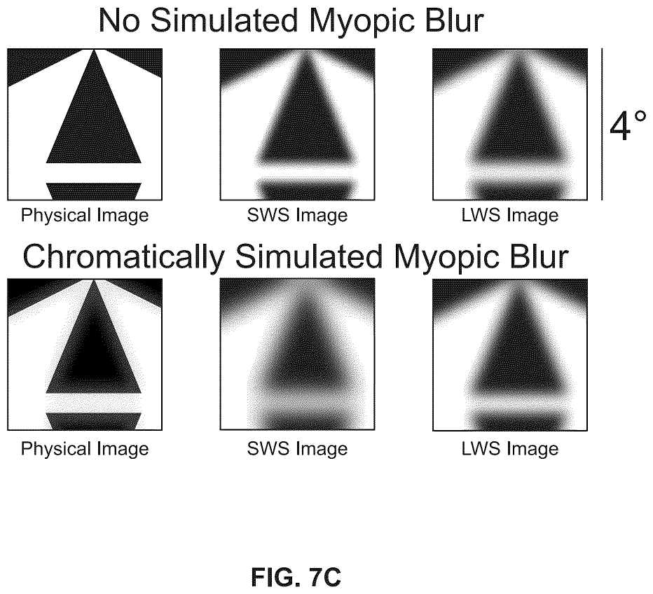

FIGS. 7A-7C illustrate the design principle for control of the emmetropization mechanism in accordance with aspects of the present disclosure. As with most computer-generated images, each pixel consists of a red, green, and blue component. FIG. 7A shows a three channel (red, green, and blue) sharp image. As shown in FIG. 7B, a high-contrast pattern such as a black-and-white pattern has the blue pixels (blue channel) selectively blurred, while the green and red pixels (green and red channel) are left undisturbed. As the blue is spread out, it leaves the white surrounding the black triangles, resulting a yellow halo outside the black triangles, and the blue bleeds into the black triangles (see, FIGS. 7B and 7C), resulting in a blue halo inside the black triangles. The same principle of selectively blurring the blue pixels can be used on other images and patterns, not just of black Maltese triangles on a white background. The result is that, when viewing this image, the shorter-wavelength cones should ‘see’ a more blurred image (see SWS image in FIG. 7C) than the longer wavelength cones (see LWS image in FIG. 6C). This would be consistent with an eye that is too long for its optics, and signal that the eye should stop or slow its rate of elongation—which would stop or slow the progression of myopia.

Although the description herein is focused on anti-myopia visual display therapy using a simulated myopic blur, it should be understood that the design principle for control of the emmetropization mechanism may also be applicable for anti-hyperopia visual display therapy using a simulated hyperopia blur. For example, rather than blurring the blue channel, the red channel may be blurred for anti-hyperopia visual display therapy.

### III. Experiments and Advantages Over Conventional Techniques

The techniques described in accordance with this disclosure may be better understood by referring to the following examples.

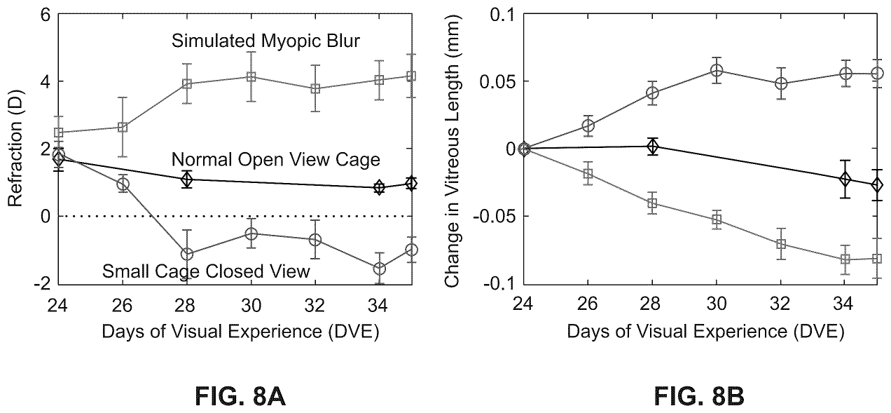

The design principle for control of the emmetropization mechanism via a simulated myopic blur was tested using as an animal model, the tree shrew, which is a diurnal mammal closely related to primates that have long been used as a model of myopia development. The results are shown in FIGS. 8A and 8B. FIG. 8A shows the refractions as a function of age. Tree shrews are born with their eyes closed so time was measured as days after eye opening (Days of Visual Experience, DVE). Refraction is measured in diopters (D). Plus diopters is hyperopic, minus diopters is myopic, and zero diopters is emmetropic (perfect focus). The black line is the mean+/−the stderr of seven normal animals raised in open cages in an environment with an extended visual view. Like non-myopic human children, tree shrews start out hyperopic and then slowly converge towards emmetropia from the hyperopic side. The red lines indicate the seven animals that at 24 DVE were removed from the colony and placed in small cubical cages 28 cm on a side internal dimension, with no external. There refractions moved significantly negative compared to the normal animals, indicating that this environment was myopiagenic. The blue lines indicate the results of eight animals that, at 24 DVE, were removed from the colony and placed in the small cage, but one wall of the cage was replaced with a video display showing the image in FIG. 7B. Not only did these animals not become myopic, they went the other way and became hyperopic-demonstrating the strong anti-myopia potency of this display. FIG. 8B is laid out similar to FIG. 8A, but it shows the change in the length of the vitreous chamber (the clear part of the eye between the lens and the retina that is the major component of eye size) length over time. As expected, relative to the normal animals, the animals that developed myopia in the closed cage had relatively greater increases in vitreous chamber depth, while the animals that developed hyperopia when viewing the anti-myopia video display, had relatively reduced vitreous chamber depths. As emmetropization in mammals occurs by alterations in the elongation of the vitreous chamber, and as it is the increase length of the eye in myopia that correlates with an increased risk of eye disease, this further illustrates the potential utility of this technique to control the progression of myopia.

Viewing these displays should be readily tolerated with minimal risks, non-invasive, simple to use (promoting compliance with treatment) and, if necessary, can be combined with other anti-myopia treatments.

In recent years, both pharmacological and optical treatments have been examined and show promise for slowing, but not eliminating, axial elongation and myopia in children. However, pharmacological treatments involve daily use of eye drops in children. The effectiveness and safety of these over extended years of use has not been examined. Similarly, current optical treatments with contact lenses involved teaching children to properly insert and remove and maintain cleanliness of contact lenses, so there is risk of corneal infections over many years of treatment. Compliance, actually wearing the contacts or using the eye drops, has been a problem. So has drop-out (stopping treatment). Despite these drawbacks, optical and pharmacological treatments are being commercially marketed in East Asia, where myopia has reached epidemic proportions, but have not received FDA approval in the U.S. The anti-myopia visual display therapy using a simulated myopic blur is an improvement over these other treatments for at least the reason that it does not involve any direct contact with eyes.

The image display should be readily presentable on standard computer monitors or large-screen televisions or, potentially, as a printed image or wallpaper, or back-lit through a transparency, or implemented as an array of physical LEDs or electroluminescent wires, or the like. As an example, a child could view a movie or other image in a small centrally located window while the anti-myopia display is presented in the rest of the screen. It has been shown that central vision is not critical for emmetropization, so a small window of a normal image in central vision should not interfere with the anti-myopia effectiveness of the image in the rest of the screen. Alternatively, the image could be presented as an overlay over an existing image, or only presented in the corners or edges of the display. If children could perform other activities during the treatment, this could greatly increase compliance. Data from the animal models strongly suggests that this therapy could be effective even for only one or two short periods per day. The exact amount of blurring of the blue pixels may be adjusted to viewing distance and monitor size according to an optical model that the inventors developed (Gawne et al., How chromatic cues can guide human eye growth to achieve good focus, Journal of Vision (2021) 21(5):11, 1-11, which is incorporated by reference herein in its entirety for all purposes). Advantageously, these techniques could revolutionize the treatment of myopia, radically reduce the incidence and severity of myopia in the human population, with concomitant increases in quality of life and reduction in sight-threatening diseases and be used routinely by hundreds of millions (if not billions) of children across the world.

### IV. Exemplary Techniques

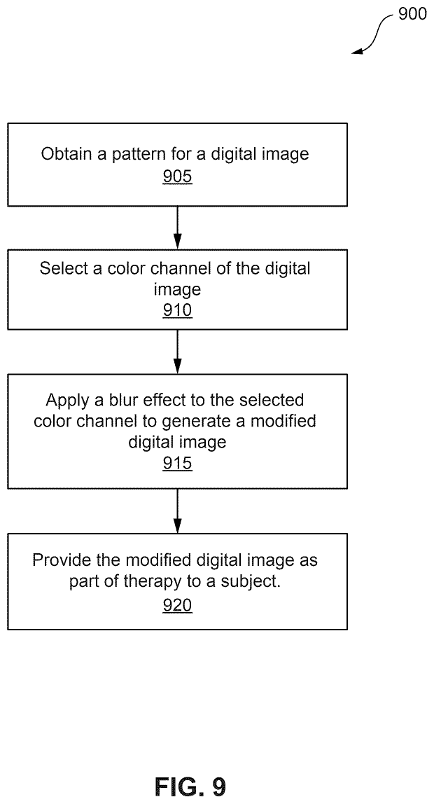

FIG. 9 shows a flowchart of a process 900 for generating an anti-myopia simulated myopic blur visual display and treating myopia using an anti-myopia simulated myopic blur visual display. The process 900 depicted in FIG. 9 may be implemented in software (e.g., code, instructions, program) executed by one or more processing units (e.g., processors, cores) of the respective systems, hardware, or combinations thereof. The software may be stored on a non-transitory storage medium (e.g., on a memory device). The process 900 presented in FIG. 9 and described below is intended to be illustrative and non-limiting. Although FIG. 9 depicts the various processing steps occurring in a particular sequence or order, this is not intended to be limiting. In certain alternative embodiments, the steps may be performed in some different order or some steps may also be performed in parallel. In certain embodiments, such as in the embodiments depicted in FIGS. 1-8B, process 900 may be performed using one or more computing systems (e.g., a personal computing device such as a cellphone, laptop, tablet, or television).

At block 905, a pattern is obtained for a digital image. Obtaining the pattern may be performed by generating a new pattern or selecting a pre-defined the pattern using a digital image application such as Adobe Photoshop®, a digital image editing tool provided as part of a health application such as an anti-myopia visual display therapy application, or the like. The pattern is a high-contrast pattern. In some instances, the contrast is tonal contrast. The tonal contrast positions light tones and darker tones next to each other and refers to the brightness of the elements within the image. If the image consists of extremely bright and dark areas, then it's considered high contrast. When it has a wide range of tones that go from pure white to pure black, it's medium contrast. No pure whites or blacks and a range of middle tones means it's low contrast. The pattern itself is not particularly limited but should contain broad spatial frequency content and have detail at various orientations, for example, a Maltese black-and-white cross pattern. In some instances, the pattern is a high-contrast pattern of objects on a solid background, which generates multiple black-white edges within the digital image. For example, a high-contrast pattern of black objects on a solid white background, which generates multiple black-white edges within the digital image; or a high-contrast pattern of white objects on a solid black background, which generates multiple black-white edges within the digital image.

At block 910, a color channel of the digital image is selected. When the digital image is opened or viewed in a digital image application or digital image editing tool, it may appear in a RGB mode, which means that it's made up of three primary colors: red, green, and blue. There are color channels for each of these colors, which can be viewed by choosing the particular channel to display the channels palette. The composite RGB channel is typically selected by default, but a user can select the Red, Green, or Blue channel individually to preview the image data for the pattern that's stored for that channel. In some instances, the color channel is selected based on a model of structure and function of an eye that demonstrates how a combination of wavelengths of light and optical defocus regulates growth of the eye. In some instances, the color channel selected is a short wavelength channel. In certain instances, the short wavelength channel is the blue channel.

At block 915, a blur effect is applied to the color channel that modifies the digital image to have a simulated blur (e.g., a simulated myopic blur). When the digital image is opened or viewed in a digital image application or digital image editing tool, the color channel may be blurred using a blurring tool. In some instances, the blurring tool implements a Gaussian blur (also known as Gaussian smoothing), which is the result of blurring the color channel of the digital image by a Gaussian function. The amount of blurring may be defined by the radius of the blur specified as a length. The length defines the width of the blur function, i.e., how many pixels on the screen blend into each other; thus, a larger value will create more blur, and a value of 0 leaves the input unchanged. The exact amount of blurring of the color channel may be adjusted based on viewing distance and/or display or image size to be viewed by a subject (e.g., a patient). In certain instances, the blur effect is applied to the color channel in a predetermined amount determined based on a viewing distance and/or display or image size to be viewed by the subject.

In certain instances, a high-contrast black-and-white pattern has the blue pixels selectively blurred, while the green and red pixels are left undisturbed. As the blue is spread out, it leaves the white surrounding the black objects, resulting a yellow halo outside the black objects, and the blue bleeds into the black objects, resulting in a blue halo inside the black objects. The same principle of selectively blurring the blue pixels can be used on other images, not just of black objects on white. The result is that, when viewing this image, the shorter-wavelength cones should ‘see’ a more blurred image than the longer wavelength cones.

At block 920, the modified digital image is provided. The providing the modified digital image may comprise rendering the modified digital image on a display of a computing device, communicating the modified digital image to a storage device or a health application such as an anti-myopia visual display therapy application, printing the modified digital image on a physical media such as paper, or a combination thereof.

In some instances, the modified digital image is provided in accordance with anti-myopia visual display therapy. The therapy may be provided by: (i) rendering the modified digital image on a display of a computing device within the visual environment of a subject, or (ii) placing the modified digital image within the visual environment of a subject, based on an optimal viewing time. The optimal viewing time may be periodically during a day (e.g., periodically during each day of the week, or every other day of the week, or on a predetermined schedule). The optimal viewing time may be determined (e.g., by a health care provider or the health application such as an anti-myopia visual display therapy application), which might be as little as a few minutes two or more times per day. In some instances, the optimal viewing time is determined based on a present level of refractive error of the eye (myopia). As an example, a child could view a movie or other image in a small centrally located window while the modified digital image is presented in the rest of the display periodically. Alternatively, the modified digital image could be presented as an overlay over an existing image periodically, or only presented in the corners or edges of the display. If children could perform other activities during the treatment, this could greatly increase compliance.

### V. Additional Considerations

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

1. A method comprising:
obtaining a pattern for a digital image;
selecting a color channel of the digital image;
applying a blur effect to the color channel that modifies the digital image to have a simulated blur; and
providing anti-myopia visual display therapy to a subject using the modified digital image, wherein the therapy comprises: (i) rendering the modified digital image on a display of a computing device within a visual environment of the subject, or (ii) placing the modified digital image within the visual environment of the subject, based on an optimal viewing time.

2. The method of claim 1, wherein the pattern is a high-contrast pattern of objects on a solid background, which generates multiple black-white edges within the digital image.

3. The method of claim 1, wherein the color channel is selected based on a model of structure and function of an eye that demonstrates how a combination of wavelengths of light and optical defocus regulates growth of the eye.

4. The method of claim 3, wherein the color channel selected is a short wavelength channel.

5. The method of claim 4, wherein the short wavelength channel is the blue channel.

6. The method of claim 1, wherein the blur effect is applied to the color channel in a predetermined amount determined based on a viewing distance and/or display or image size to be viewed by the subject.

7. The method of claim 1, wherein the modified digital image is rendered on the display of the computing device in combination with other images unrelated to the therapy.

8. The method of claim 1, wherein the optimal viewing time is determined based on a present level of refractive error of an eye of the subject.

9. The method of claim 8, wherein the optimal viewing time is periodically through a day.

10. A system comprising: one or more processors; and a memory coupled to the one or more processors, the memory storing a plurality of instructions executable by the one or more processors, the plurality of instructions comprising instructions that when executed by the one or more processors cause the one or more processors to perform the following operations:
obtaining a pattern for a digital image;
selecting a color channel of the digital image;
applying a blur effect to the color channel that modifies the digital image to have a simulated blur; and
providing anti-myopia visual display therapy to a subject using the modified digital image, wherein the therapy comprises rendering the modified digital image on a display of the system within a visual environment of the subject.

11. The system of claim 10, wherein the pattern is a high-contrast pattern of objects on a solid background, which generates multiple black-white edges within the digital image.

12. The system of claim 10, wherein the color channel is selected based on a model of structure and function of an eye that demonstrates how a combination of wavelengths of light and optical defocus regulates growth of the eye.

13. The system of claim 12, wherein the color channel selected is a short wavelength channel.

14. The system of claim 13, wherein the short wavelength channel is the blue channel.

15. The system of claim 10, wherein the blur effect is applied to the color channel in a predetermined amount determined based on a viewing distance and/or display or image size to be viewed by the subject.

16. A non-transitory computer-readable memory storing a plurality of instructions executable by one or more processors, the plurality of instructions comprising instructions that when executed by the one or more processors cause the one or more processors to perform the following operations:
obtaining a pattern for a digital image;
selecting a color channel of the digital image;
applying a blur effect to the color channel that modifies the digital image to have a simulated blur; and
providing anti-myopia visual display therapy to a subject using the modified digital image, wherein the therapy comprises rendering the modified digital image on a display of the system within a visual environment of the subject.

17. The non-transitory computer-readable memory of claim 16, wherein the pattern is a high-contrast pattern of objects on a solid background, which generates multiple black-white edges within the digital image.

18. The non-transitory computer-readable memory of claim 16, wherein the color channel is selected based on a model of structure and function of an eye that demonstrates how a combination of wavelengths of light and optical defocus regulates growth of the eye.

19. The non-transitory computer-readable memory of claim 18, wherein the color channel selected is a short wavelength channel, and wherein the short wavelength channel is the blue channel.

20. The non-transitory computer-readable memory of claim 16, wherein the blur effect is applied to the color channel in a predetermined amount determined based on a viewing distance and/or display or image size to be viewed by the subject.
